(19) United States
(12) Patent Application Publication
(10) Pub. No.: US 2021/0069260 A1
(43) Pub. Date: Mar. 11, 2021

(54) Use of Bacteroides fragilis in preparing drugs for inducing at least one of proliferation and accumulation of &#947;&#948; T cells

(71) Applicant: REVAISSANT (SHENZHEN) BIOSCIENCES CO., LTD., Shenzhen, Guangdong (CN)

(72) Inventor: Gucheng Zeng, Guangzhou City (CN)

(21) Appl. No.: 16/772,189

(22) PCT Filed: Jan. 23, 2018
(86) PCT No.: PCT/CN2018/073687

§ 371 Date: Jun. 12, 2020

(51) Int. Cl. A61K 35/741; A61P 37/04; A23L 33/135

(52) U.S. Cl. CPC A61K 35/741; A61K 2035/115; A23L 33/135; A61P 37/04

(57) ABSTRACT

Provided is a use of Bacteroides fragilis in preparing a drug for inducing at least one of proliferation and accumulation of γδ T cells. Also provided is a use of Bacteroides fragilis in preparing a drug for enhancing γδ T cell function.

### CROSS-REFERENCE TO RELATED APPLICATIONS

This application claims priority to PCT Application No. PCT/CN2018/073687, having a filling date of Jan. 23, 2018, which is based on Chinese Application No. 201711316873.3, having a filling date of Dec. 12, 2017, the entire contents both of which are incorporated herein by reference.

### FIELD OF TECHNOLOGY

The following belongs to the technical field of immunology. The present disclosure relates to a composition capable of inducing a proliferation, an accumulation and an enhanced effector function of γδ T cells (especially TCR γδ-strong positive T cells) in infectious diseases and/or tumors. The composition comprises Bacteroides fragilis or physiological active ingredient obtained from the Bacteroides fragilis, or comprises a complex using Bacteroides fragilis as the active ingredient. The present disclosure further relates to a method of inducing the proliferation of γδ T cells and enhancing their effector function in infectious diseases and/or tumors.

### BACKGROUND

Hundreds of symbiotic microorganism species live in the gastrointestinal tract, and closely interact with the immune system of host. It is demonstrated by the results of researches with the germfree animals (GF) that the symbiotic microorganism plays a great role in formation of mucosal immune system, such as histogenesis of peyer' s patch (PP) and isolated lymphoid follicles (ILF), secretion of antimicrobic peptide from the epithelial cells, and accumulation of unique lymphocyte in mucosa tissues, and the unique lymphocyte comprises plasmocyte generating immunoglobulin A, intraepithelial lymphocyte, positive T cells (Th17) generating IL-17 and NK cells generating IL-22. Therefore, the presence of intestinal bacteria enhances the protection effect of mucosa, and provides a strong immune reaction against the invading pathogenic microorganism for the host. In another aspect, the mucosa immune system maintains an anergy to dietary antigens and harmless microorganism. Therefore, a dysregulation (disorder of intestinal ecosystem) of the cross-talk between the symbiotic bacteria and immune system may cause an immune overreaction to the environmental antigens, thereby leading to an inflammatory bowel disease (IBD).

It is demonstrated by the recent research results that the isolated symbiotic bacteria control the differentiation of specific immune cells in the mucosal immune system. Segmented filamentous bacteria are intestinal symbiotic bacteria of mouse, and they can induce mucosal Th17 cells to respond and thereby enhance a resistance against the gastrointestinal pathogen infection of the host, as shown by the research. Further, it is known that the intestinal inflammation can be repressed by a short chain fatty acid obtained by several types of symbiotic bacteria.

Bacteroides fragilis (B. fragilis) is an obligate anaerobic bacterium which is Gram-negative, rod-shaped, and non-motile, having obtuse and hyperchromatic ends as well as a capsule, without spores. The Bacteroides fragilis can be classified into an enterotoxigenic type and a non-enterotoxigenic type. As a part of the normal intestinal flora of humans and animals, Bacteroides fragilis mainly exists in the colon, and besides, it can also colonize and grow in the respiratory tract, the gastrointestinal tract and the urogenital tract.

The γδ T cell is a special cellular subgroup of T lymphocyte, and has a unique structure and biological function. Unlike the general αβ T cell, the γδ T cell surface receptor is consisted of two glycoprotein chains, i.e. γ chain and δ chain. Such cells, capable of recognizing the antigens without presentation of histocompatibility antigen molecules on the cellular surface, can directly recognize the proteins, peptides, and non-peptide antigens, which is an important biological characteristic of γδ T cell. The γδ T cell plays an important role in the specific and nonspecific immune system, although it accounts for a smaller proportion in the peripheral blood and lymphoid organ, compared with αβ T cell. The γδ T cell can proliferate and accumulate greatly under the stimulation of antigen, and migrate rapidly to the non-lymphoid organ to take anti-infection and anti-tumor effect, preventing the invasion of pathogens comprising bacteria, fungus, virus and parasite, and playing an important role in the immunological surveillance of tumor, maintenance of tissue homeostasis and recovery of tissues during the inflammatory response and post-inflammation. However, it is still unclear whether and how the Bacteroides fragilis affect the immunological function of γδ T cell, and it is also unclear how to efficiently achieve the proliferation, and/or accumulation and enhanced effector function of γδ T cell.

Therefore, it is significant to treat and/or prevent infection caused by tumors, tuberculosis, drug-resistant bacteria, fungus, virus, chlamydia, etc., by illustrating that the Bacteroides fragilis can affect the proliferation of γδ T cell and enhance its effector function, thereby enhancing the immune function of γδ T cell.

### SUMMARY

An aspect relates to a composition comprising Bacteroides fragilis, or a physiological active substance or a complex obtained from the Bacteroides fragilis in infectious diseases and/or tumors to induce a proliferation and an effector function of γδ T cells.

Another aspect relates to a method of inducing and enhancing the effector function of γδ T cells in infectious diseases and/or tumors.

The inventors of the present invention find that it is possible to adjust the immunologic response of T cells in infectious diseases and/or tumors, by improving the proliferation and effector function of γδ T cells and/or TCR γδ-strong positive T cells with Bacteroides fragilis. Specifically, the inventors find that the Bacteroides fragilis, or the physiological active substance or the complex obtained from the Bacteroides fragilis in infectious diseases and/or tumors can promote the systematic proliferation of γδ T cells and/or TCR γδ-strong positive T cells in lymphoid organs and increased expression of IFN-γ in γδ T cells, thereby the anti-infection and anti-tumor effect of T cells can be enhanced. Thus the present invention is achieved.

More specifically, the present disclosure has following aspects:

Another aspect relates to a use of Bacteroides fragilis in preparing drugs for inducing the proliferation and/or accumulation of γδ T cells.

In an aspect, the Bacteroides fragilis is selected from any one of living Bacteroides fragilis; genetically recombined, altered or modified, attenuated, chemically treated, physically treated or inactivated Bacteroides fragilis; Bacteroides fragilis lysate; and/or Bacteroides fragilis culture supernatant.

In another aspect, the γδ T cell has a phenotypic characteristic of TCR γδ+ (also called as γδTCR+, TCR-γδ+, or γδ-TCR).

Another aspect relates to a composition used for inducing the proliferation and/or accumulation of γδ T cells, and the composition comprises Bacteroides fragilis as an active ingredient.

In another aspect, the Bacteroides fragilis is selected from any one of living Bacteroides fragilis; genetically recombined, altered or modified, attenuated, chemically treated, physically treated or inactivated Bacteroides fragilis; Bacteroides fragilis lysate; and/or Bacteroides fragilis culture supernatant.

In another aspect, the composition is any one of pharmaceutical compositions, foods, health products, or food additives.

Another aspect relates to a use of Bacteroides fragilis in preparing drugs for enhancing functions of γδ T cells.

In an aspect, the Bacteroides fragilis is selected from any one of living Bacteroides fragilis; genetically recombined, altered or modified, attenuated, chemically treated, physically treated or inactivated Bacteroides fragilis; Bacteroides fragilis lysate; and/or Bacteroides fragilis culture supernatant.

Another aspect relates to a composition used for enhancing functions of γδ T cells, and the composition comprises Bacteroides fragilis as an active ingredient.

In an aspect, the Bacteroides fragilis is selected from any one of living Bacteroides fragilis; genetically recombined, altered or modified, attenuated, chemically treated, physically treated or inactivated Bacteroides fragilis; Bacteroides fragilis lysate; and/or Bacteroides fragilis culture supernatant.

In an aspect, the composition is any one of pharmaceutical compositions, foods, health products, or food additives.

Specifically, another aspect relates to a method of inducing a proliferation, migration and enhanced effector function of γδ T cells in an individual (if there is an individual in need, such as in need of inducing proliferation and/or accumulation and enhanced effector function), and the method comprises steps of:
(a) administrating a composition to the individual, and the composition comprises at least one of living Bacteroides fragilis; genetically recombined, altered or modified, attenuated, chemically treated, physically treated or inactivated Bacteroides fragilis; Bacteroides fragilis lysate; and/or Bacteroides fragilis culture supernatant; as an active ingredient;
(b) inducing the effector function of γδ T cells: the day administering the active ingredient is deemed as day 0; the active ingredient is administered for induction every 3 days, and the mouse is infected by pathogen or transplanted with tumors after two weeks; the active ingredient is kept being administered and the mouse is dissected at day 30 to detect the content of γδ T cell in the mouse;
(c) detecting the content of γδ T cell: the proportion of γδ T cell in the cultured cells is detected though flow cytometry, by taking TCR γδ, CD4, and CD8 as the molecular marker, and TCR γδ+ as the phenotypic characteristic of γδ T cells;
(d) detecting the effector function of γδ T cell: the proportion of γδ T cell expressing interferon (IFN-γ) is analyzed through flow cytometry.

According to the method, the detection of TCR γδ+ is used as an indicator of proliferation or accumulation of γδ T cells in the individual.

According to the method, when referring to the γδ T cell with enhanced function, the enhanced function means enhanced anti-infection and anti-tumor effect in the body.

According to the method, when referring to the γδ T cell and/or TCR γδ-strong positive T cell with enhanced anti-infection effect, the enhanced anti-infection effect means increased generation of interferon-γ (IFN-γ) of T cell.

According to the method, enhancing the effector function of γδ T cell comprises administering infectious pathogen or transplanting tumor to an individual.

The composition of the present disclosure contains Bacteroides fragilis as an active ingredient, and is excellent in inducting the proliferation and accumulation of γδ T cells, or promoting their effector functions. The immunity in living organisms can be improved by administering the composition of the present disclosure as a pharmaceutical product or ingesting the composition as food or beverage. Therefore, the composition of the present disclosure may be used for, for example, prevention or treatment of autoimmune diseases or allergic diseases, etc. Besides, the composition can be ingested easily and routinely by a healthy individual if the composition is contained in a healthy diet, such as food or beverage, thereby the proliferation or accumulation of γδ T cell and/or especially TCR γδ-strong positive T cell can be induced to improve the immune function.

### BRIEF DESCRIPTION

Some of the embodiments will be described in detail, with reference to the following figures, wherein like designations denote like members, wherein:

FIG. 1 is a schematic flow chart of an experiment for detecting the enhanced proliferation and accumulation, or promoted effector function of γδ T cells caused by Bacteroides fragilis in an infectious disease;

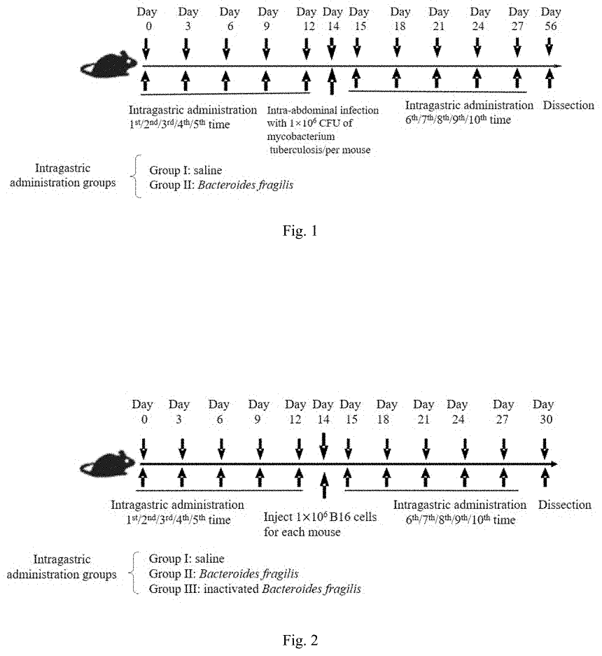

FIG. 2 is a schematic flow chart of an experiment for detecting the enhanced proliferation and accumulation, or promoted effector function of γδ T cells caused by Bacteroides fragilis or inactivated Bacteroides fragilis in a tumor;

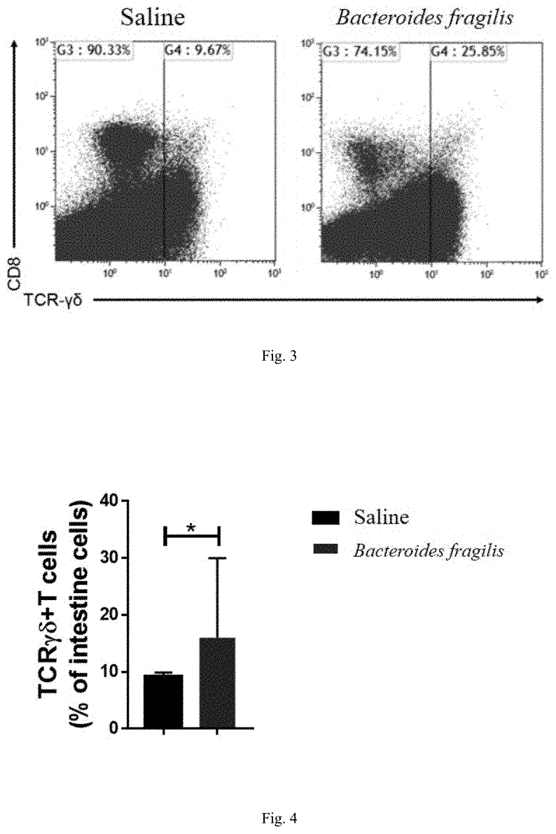

FIG. 3 shows a typical flow cytometric analysis graph of one mycobacterium tuberculosis-infected mouse in each group after these mice are administered with Bacteroides fragilis, wherein the right quadrant represents TCR γδ-positive T cell (TCR γδ+ T cell), and the numbers in the right quadrant represents the proportion of TCR γδ+ T cells in overall intestinal cells;

FIG. 4 is a statistic analysis graph of percentages of TCR γδ+ T cells in overall intestinal cells of mycobacterium tuberculosis-infected mice being administered with Bacteroides fragilis;

FIG. 5 shows a typic flow cytometric analysis graph of one melanoma-transplanted mouse in each group after these mice are administered with Bacteroides fragilis and inactivated Bacteroides fragilis respectively, wherein the right quadrant represents TCR γδ+ T cells, and the numbers in the right quadrant represent the proportions of TCR γδ+ T cells in overall intestinal cells;

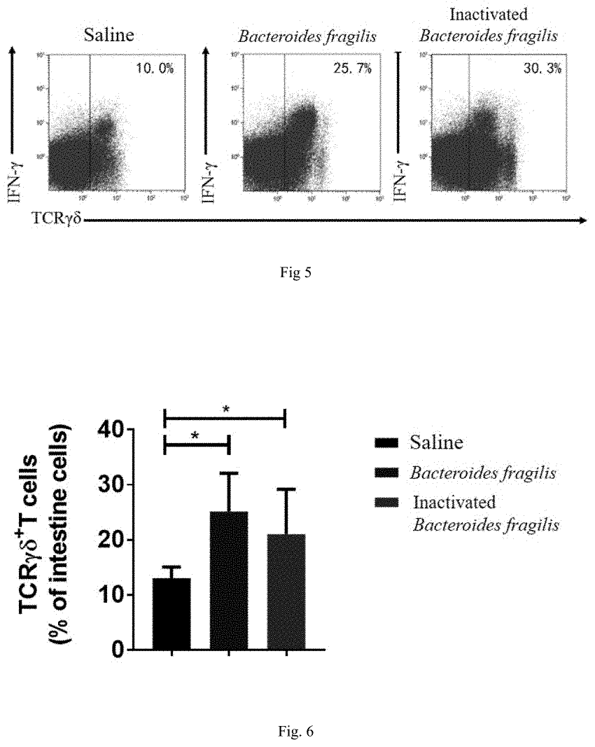

FIG. 6 is a statistic analysis graph of percentages of TCR γδ+ T cells in overall intestinal cells of melanoma-transplanted mice being administered with Bacteroides fragilis or inactivated Bacteroides fragilis;

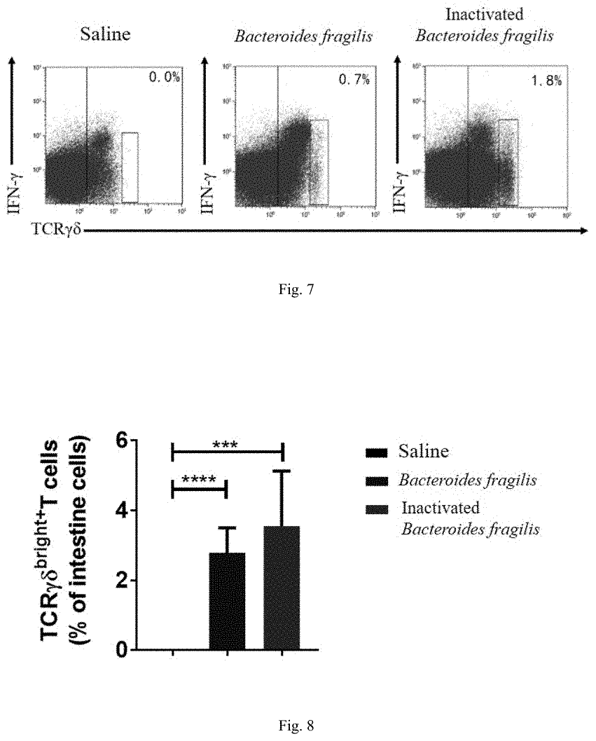

FIG. 7 shows a typic flow cytometric analysis graph of TCR γδ-strong positive T cells of one melanoma-transplanted mouse in each group after these mice are administered with Bacteroides fragilis and inactivated Bacteroides fragilis respectively, wherein the right quadrants represent TCR γδ+ T cells, the cells restrained in the rectangular frame are TCR γδ-strong positive (TCR γδ^{bright+}) T cells, and the numbers in the right quadrant indicate the percentages of TCR γδ-strong positive (TCR γδ^{bright+}) T cells in the overall intestinal cells;

FIG. 8 is a statistic analysis graph of percentages of TCR γδ-strong positive T cells in the overall intestinal cells of melanoma-transplanted mice being administered with Bacteroides fragilis or inactivated Bacteroides fragilis;

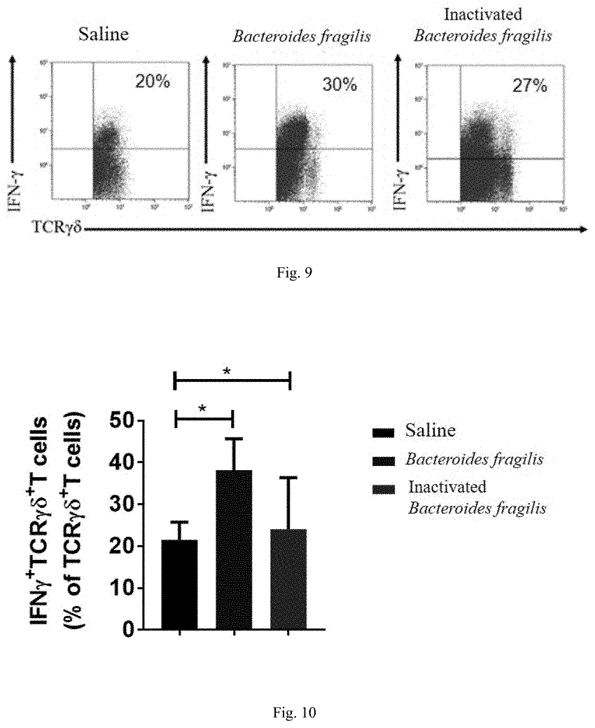

FIG. 9 shows a typic flow cytometric analysis graph of expression of IFN-γ in TCR γδ+ T cells of one melanoma-transplanted mouse in each group after these mice are administered with Bacteroides fragilis and inactivated Bacteroides fragilis respectively, wherein the numbers in the right quadrant indicate the percentages of TCR γδ+ IFN-γ+ (expressing both TCR γδ and IFN-γ) in the overall TCR γδ+ T cells;

FIG. 10 shows a statistic analysis graph of expression of IFN-γ in TCR γδ⁺ T cells of the melanoma-transplanted mice being administered with Bacteroides fragilis or inactivated Bacteroides fragilis;

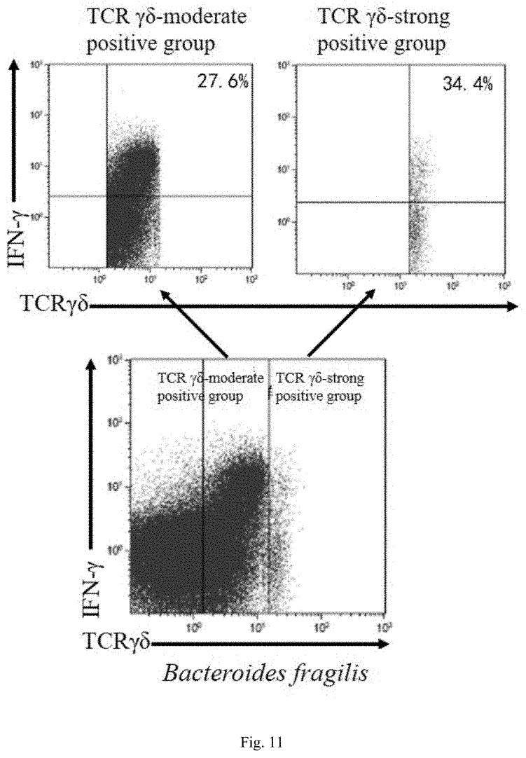

FIG. 11 shows a typic flow cytometric analysis graph showing expression of IFN-γ in TCR γδ-moderately positive (TCR γδ-moderate positive for short, TCR γδ^{medium+}) T cells and TCR γδ-strong positive T cells of one melanoma-transplanted mouse in each group after these mice are administered with Bacteroides fragilis, wherein the numbers in the right quadrant represent the percentages of TCR γδ+ IFN-γ+ T cells in TCR γδ-moderate positive T cells or TCR γδ-strong positive T cells accordingly;

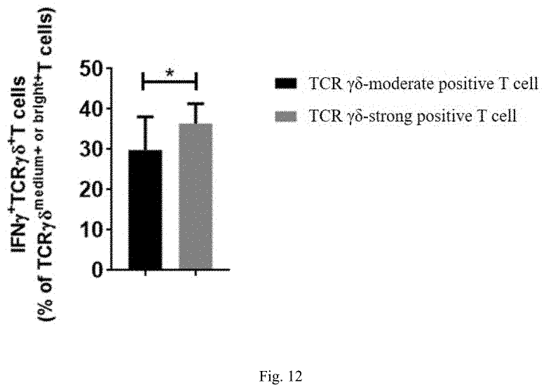

FIG. 12 is a statistic analysis graph of expression of IFN-γ in TCR γδ^{medium+}) T cells and TCR γδ^{bright+} T cells of melanoma-transplanted mice being administered with Bacteroides fragilis;

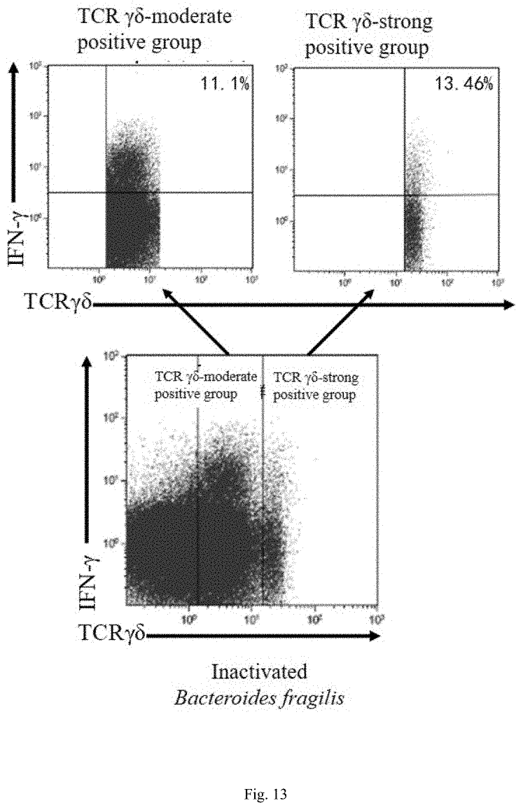

FIG. 13 shows a typic flow cytometric analysis graph showing expression of IFN-γ in TCR γδ^{medium+} T cells and TCR γδ^{bright+} T cells of one melanoma-transplanted mouse in each group after these mice are administered with inactivated Bacteroides fragilis, wherein the numbers in the right quadrant represent the percentages of TCR γδ+ IFN-γ+ T cells in TCR γδ-moderate positive T cells or c accordingly; and

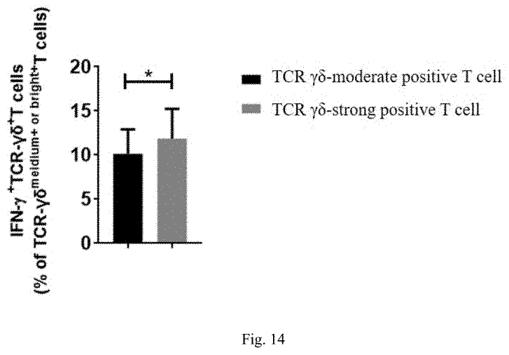

FIG. 14 is a statistic analysis graph of expression of IFN-γ in TCR γδ^{medium+} T cells and TCR γδ^{bright+} T cells of melanoma-transplanted mice being administered with inactivated Bacteroides fragilis.

### DETAILED DESCRIPTION

The present disclosure will be further described below with reference to the detailed embodiments. It should be pointed out that Bacteroides fragilis for enhancing the proliferation and effector function of γδ T cells and/or TCR γδ-strong positive T cells (TCR γδ^{bright+} T cells) or a pharmaceutical composition, a food, a health product and a food additive containing the Bacteroides fragilis of the present disclosure can be applied to the indications and exhibit the functions described above, after they are administered to the subject. The tests have been done in all dosage forms within the scope of the present disclosure. In the following, only small parts are described in the examples just for illustration, however, it should not be construed as a limitation of the disclosure.

The present disclosure provides a composition of Bacteroides fragilis for inducing the proliferation and accumulation of the γδ T cells and/or TCR γδ-strong positive T cells, or promoting their effector function, and the composition comprises at least one of living Bacteroides fragilis; genetically recombined, altered or modified, attenuated, chemically treated, physically treated or inactivated Bacteroides fragilis; Bacteroides fragilis lysate; and/or Bacteroides fragilis culture supernatant, as an active ingredient.

“The Bacteroides fragilis”, which is the active ingredient of the composition of the present disclosure, can be any bacterium capable of inducing the proliferation and accumulation of the γδ T cells and/or TCR γδ-strong positive T cells, or promoting their effector function, without any special limitation. The Bacteroides fragilis can be applied to the composition of the present disclosure alone or in combination with other bacteria.

In the present disclosure, “γδ T cell” or “γδ T cells” means T cell or T cells express the TCR γ and/or δ chain in the lymphocyte participating in immune response.

In the present disclosure, the method of enhancing the function of T cells comprises steps of promoting the proliferation, enhancement of effector function and expressing IFN-γ. In addition, in the present disclosure, the T cells having enhanced function are cells obtained through the methods of enhancing the function of T cells in the present disclosure. In the present disclosure, the method of enhancing the function of T cells may improve the effector function of T cells, comprising increasing the proliferation rate of T cells, increasing the production of cytokines, or modifying cytotoxicity. The T cells with enhanced function obtained in the present disclosure may be used for treating or preventing cancers, infectious diseases or autoimmune diseases.

The present disclosure will be further described in detail below with reference to the embodiments. However, the present disclosure is not limited to the following embodiments.

### EXAMPLE 1 BACTEROIDES FRAGILIS CULTURE

### Culture Method

Step 1: A lyophilized preserved Bacteroides fragilis strain was taken, then 200 μL culture medium of brain heart infusion broth (BHI) was added to reconstitute it. 20 μL of bacterial solution was streaked on the blood agar plate. Then the plate was placed in a biochemical incubator after an air exhaust processing by an anaerobic jar gassing system, and cultured in anaerobic environment at 37° C. for 48 hours;

Step 2: Monoclonal colony was selected and inoculated in 10 mL BHI, followed by being cultured in anaerobic environment at 37° C. for 12 hours;

Step 3: 1% (v/v) of strain was inoculated in 500 mL BHI in a flask and cultured in anaerobic environment at 37° C. for 48 hours.

Step 4: The bacterial solution was collected and centrifuged at 6000 rpm for 10 min. The bacterial sludge was washed twice with saline, and was finally reconstituted with saline for later use. The viable bacteria were counted.

### EXAMPLE 2 EXPERIMENT OF INDUCING ENHANCED EXPRESSION of γδ TCR, PROLIFERATION, ACCUMULATION AND EFFECTOR FUNCTION OF γδ T CELLS IN MYCOBACTERIUM TUBERCULOSIS-IMMUNE MICE BY BACTEROIDES FRAGILIS

### 1. Culture Method

The culture method of Bacteroides fragilis is the same as that in Example 1.

### 2. Sample Preparation

1) Preparation of Living Bacteroides fragilis ZY-312

### Culture Method:

Step 1: A lyophilized preserved strain was taken, and then 200 μL culture medium of brain heart infusion broth (BHI) was added to reconstitute it. Then 20 μL bacterial solution was pipetted, and was streaked on the blood agar plate. The plate was placed in a biochemical incubator after an air exhaust processing by an anaerobic jar gassing system, and then was cultured in anaerobic environment at 37° C. for 48 hours;

Step 2: Monoclonal colony was selected and inoculated in 10 mL BHI, and followed by being cultured in anaerobic environment at 37° C. for 12 hours;

Step 3: 1% (v/v) of strain was inoculated in 500 mL TSB in a flask, and cultured in anaerobic environment at 37° C. for 48 hours;

Step 4: The bacterial solution was taken and centrifuged at 6000 rpm for 10 minutes. The bacterial sludge was washed twice with saline, and was finally reconstituted with saline for later use. The viable bacteria were counted.

2) Inactivated Bacteroides fragilis

The bacterial solution was heated in a water bath at 70° C. for 30 minutes to obtain an inactivated bacterial solution.

3) Lysate of Bacteroides fragilis

The culture bacterial solution of Bacteroides fragilis was ultrasonically crushed with an ultrasonic disruptor, being crushed for 2 seconds and pausing for 5 seconds, lasting for 20 minutes, to obtain a lysate of Bacteroides fragilis.

4) Culture Supernatant Fluid of Bacteroides fragilis

The culture bacterial solution of Bacteroides fragilis was centrifuged with a centrifuge at 6000 rpm for 10 minutes, to obtain a culture supernatant fluid of Bacteroides fragilis.

3. Experiment of Inducing Enhanced Expression and Effector Function of γδ T Cells in Mycobacterium Tuberculosis-Infected Mice by Bacteroides fragilis.

FIG. 1 is a schematic flow chart of an experiment for detecting the enhanced proliferation and accumulation, or promoted effector function of γδ T cells in an infectious disease by Bacteroides

Experimental animals: twenty-four C57BL/6 mice, 3 to 4 weeks old, in good mental state, were purchased from the Experimental Animal Center of Sun Yat-sen University. The mice were randomly divided into 2 groups, 12 mice for each group, and the 2 groups were the control group and the living bacteria gavage group respectively. 1×10⁹ CFU of Bacteroides fragilis and control solution were respectively administered intragastrically to the two groups of mice for 2 weeks, and then the mice were infected with mycobacterium tuberculosis, followed by a continued intragastrical administration for 2 weeks, during which 1×10⁹ CFU of Bacteroides fragilis and control solution were respectively administered intragastrically to the two groups of mice every 3 days. 6 weeks after the mice were infected with mycobacterium tuberculosis, they were dissected to collect cells respectively. FCM (flow cytometry) was conducted to detect and analyze the content of γδ T cells and the expression of cytokines.

### EXAMPLE 3 EXPERIMENT OF INDUCING ENHANCED EXPRESSION OF γδ TCR, PROLIFERATION, ACCUMULATION AND EFFECTOR FUNCTION OF γδ T CELLS IN MELANOMA-TRANSPLANTED MICE BY BACTEROIDES FRAGILIS

### 1. Culture Method

The culture method of Bacteroides fragilis is the same as that in Example 1.

### 2. Sample Preparation

The sample preparation is the same as that in Example 2.

3. Experiment of Inducing Enhanced Expression and Effector Function of γδ T Cells in Melanoma-Transplanted Mice by Bacteroides fragilis.

FIG. 2 is a schematic flow chart of an experiment for detecting the enhanced proliferation and accumulation, or promoted effector function of γδ T cells caused by Bacteroides fragilis in a tumor;

Experimental animals: thirty-six C57BL/6 mice, 3 to 4 weeks old, in good mental state, were purchased from the Experimental Animal Center of Sun Yat-sen University. The mice were randomly divided into 3 groups, 12 mice for each group, and the 3 groups were the control group, the living bacteria gavage group and the inactivated bacteria gavage group respectively. 1×10⁹ CFU of Bacteroides fragilis and control solution were respectively administered intragastrically to the three groups of mice for 2 weeks, and the body weights of the mice were measured daily. The murine tumor (melanoma) cells B16 were digested with TE after they were grown to logarithmic phase, and the medium was used for neutralizing. The cells were collected through centrifugation, and then washed twice with DPBS to remove the residual serum. The cells were resuspended with DPBS and counted. 1×10⁶B16 cells were inoculated subcutaneously into the right armpit of each mouse, and the intragastrical administration was continued. After 2 weeks, the tumor-bearing mice were killed to collect cells, and FCM was conducted to detect and analyze the expression of TCR γδ, content of γδ T cells and expression of cytokines.

### EXAMPLE 4 DETECTION OF CONTENT AND EFFECTOR FUNCTION OF γδ T CELLS THROUGH FCM

(1) The cells were collected in tubes, and there were 10⁷ cells in each tube; PBS buffer solution was used to wash twice, followed by centrifugation at 300 g for 6 min; after the supernate was removed, 40 μL PBS buffer solution was used to resuspend the cells;
(2) Anti-TCR γδ-FITC monoclonal antibody was added and mixed uniformly, followed by an incubation in dark at 4° C. for 30 minutes;
(3) PBS buffer solution was used to wash twice, followed by centrifugation at 300 g for 6 min, and then the supernate was removed;
(4) 1 ml of fixation buffer solution (fixation/permeabilization buffer solution) was added and mixed uniformly, followed by an incubation in dark at 4° C. for 30 minutes;
(5) Wash buffer solution (permeabilization wash buffer solution) was used to wash twice, followed by centrifugation at 300 g for 6 min, and then the supernate was removed;
(6) 40 μL of FACS buffer solution was used to resuspend the cells, and anti-IFN-γ-APC monoclonal antibody was added and mixed uniformly, followed by an incubation in dark at 4° C. for 30 minutes;
(7) Wash buffer solution was used to wash twice, followed by centrifugation at 300 g for 6 min, and then after the supernate was removed, PBS buffer solution was added to resuspend cells;
(8) A flow cytometer was used to test, by taking TCR γδ+ as a phenotypic characteristic of γδ T cell, and TCR γδ+IFNγ+ as a characteristic of enhanced anti-infectious effect of γδ T cell.

### Results analysis

Typical flow testing results for each mouse and statistical results of mice in each group are shown in FIGS. 3-14.

FIG. 3 shows a typic flow cytometric analysis graph of one mycobacterium tuberculosis-infected mouse in each group after these mice are administered with Bacteroides fragilis, the numbers in the right quadrant represent the percentages of TCR γδ+ T cells in overall intestinal cells. As can be seen from the flow cytometric analysis graph, Bacteroides fragilis increases the relative amount of γδ T cells by 2-3 times compared with the saline control group. FIG. 4 is a statistic analysis graph of percentages of TCR γδ+ T cells in overall intestinal cells of mycobacterium tuberculosis-infected mice being administered with Bacteroides fragilis. As can be seen from the statistic graph, Bacteroides fragilis significantly increases the relative amount of TCR γδ+ T cells compared with the saline control group. In the statistical analysis graph, “*” represents student t-test p<0.05. p<0.05, the difference has statistical significance. There are twelve mice in each treatment group. It is indicated from above results that the amount of γδ T cells increases significantly after an administration of Bacteroides fragilis, compared to the saline control group, for an infectious disease (FIGS. 2-3).

FIG. 5 shows a typic flow cytometric analysis graph of one melanoma-transplanted mouse in each group after these mice are administered with Bacteroides fragilis, inactivated Bacteroides fragilis and saline control respectively, and the numbers in the right quadrant represent the percentages of TCR γδ+ T cells in overall intestinal cells. As can be seen from the flow cytometric analysis quadrant diagram, the Bacteroides fragilis and inactivated Bacteroides fragilis increase the relative amount of γδ T cells by 2-3 times compared with the saline control group. FIG. 6 is a statistic analysis graph of percentages of TCR γδ+ T cells in overall intestinal cells of melanoma-transplanted mice being administered with Bacteroides fragilis, inactivated Bacteroides fragilis and saline control respectively. As can be seen from the statistic graph, the Bacteroides fragilis and inactivated Bacteroides fragilis significantly increase the relative amount of TCR γδ+ T cells compared with the saline control group. In the statistical analysis graph, * represents student t-test p<0.05. p<0.05, the difference has statistical significance. There are twelve mice in each treatment group.

In order to further illustrate the effect of Bacteroides fragilis and inactivated Bacteroides fragilis on the expression of TCR γδ and the function of γδ T cells, the flow cytometric intensity of the expression of TCR γδ will be analyzed. The higher the expression of TCR γδ is, the higher the flow cytometric intensity is, under the same flow cytometric voltage. FIG. 7 shows a typic flow cytometric analysis graph of one melanoma-transplanted mouse in each group after these mice are administered with Bacteroides fragilis, and as seen from the quadrant diagram of flow cytometric analysis, a group of TCR γδ-strong positive (TCR γδ^{bright+}) cells appear in overall TCRγδ+ T cells after the mice are treated with Bacteroides fragilis or inactivated Bacteroides fragilis, compared with the saline control group. The numbers in the right quadrant indicate the percentages of TCR γδ^{bright+} T cells restrained in the rectangular frame in the overall intestinal cells. FIG. 8 is a statistic analysis graph of percentages of TCR γδ^{bright+} T cells in the overall intestinal cells of the melanoma-transplanted mice being administered with Bacteroides fragilis. As can be seen from the statistic graph, the Bacteroides fragilis and inactivated Bacteroides fragilis significantly induce a group of TCR γδ^{bright+} T cells compared with the saline control group. In the statistical analysis graph, **** represents student t-test p<0.0001, *** represents p<0.001. p<0.05, the difference has statistical significance. There are twelve mice in each treatment group. The results indicate that the effect of promoting the proliferation of γδ T cells and enhancing effector function by Bacteroides fragilis not only presents in the pathogen-infected mice as shown in FIGS. 3 and 4, but also exist in tumors. As shown in FIGS. 5-8, after the tumor is transplanted, the proportion of γδ T cells increases by about 2 to 3 times or above and the portion of TCR γδ-strong positive T cells increases by about 700-1800 times or above, when they are induced by Bacteroides fragilis and inactivated Bacteroides fragilis, compared with the control group.

FIG. 9 shows a typic flow cytometric analysis graph of expression of IFN-γ in TCR γδ+ T cells of one melanoma-transplanted mouse in each group after these mice are administered with Bacteroides fragilis. The numbers in the right quadrant represent the percentages of TCR γδ+ IFN-γ+ (expressing both TCR γδ and IFN-γ). As can be seen from the flow cytometric analysis quadrant diagram, the Bacteroides fragilis and inactivated Bacteroides fragilis significantly increase the expression of IFN-γ in γδ T cells. FIG. 10 shows a statistic analysis of expression of IFN-γ in TCR γδ⁺ T cells of the melanoma-transplanted mice being administered with Bacteroides fragilis or inactivated Bacteroides fragilis. As can be seen from the statistic graph, the Bacteroides fragilis and inactivated Bacteroides fragilis significantly increase the expression of IFN-γ in γδ T cells, compared with the saline control group. In the statistical analysis graph, * represents student t-test p<0.05. p<0.05, the difference has statistical significance. There are twelve mice in each treatment group. It can be seen that the proportion of IFN-γ+ (expressing IFN-γ) in δ T cells increases significantly (FIGS. 9-10) compared with the control group. IFN-γ is an important anti-tumor immunocyte effector molecule. A greater amount of IFN-γ and/or higher percentage of IFN-γ+ T cells indicate a stronger effector function of T cells.

The results of FIGS. 9-10 demonstrate that the Bacteroides fragilis and inactivated Bacteroides fragilis can significantly enhance the effector function of γδ T cells.

In order to further illustrate the significance of generation of TCR γδ^{bright+} T cells induced by Bacteroides fragilis or inactivated Bacteroides fragilis, the TCR γδ+ T cells are divided into two groups of TCR γδ-moderately positive (TCR γδ-moderate positive, for short, TCR γδ^{medium+}) T cells and TCR γδ-strong positive (TCR γδ^{bright+})T cells, according to the flow cytometric intensity of the expression of TCR γδ, and the expression of IFN-γ of TCR γδ^{medium+} T cells and TCR γδ^{bright+} T cells are compared. The cell expressing IFN-γ is defined as IFN-γ+.

FIG. 11 shows a typic flow cytometric analysis graph of expression of IFN-γ in TCR γδ-moderate positive (TCR γδ^{medium+}) T cells and TCR γδ-strong positive (TCR γδ^{bright+}) T cells of one melanoma-transplanted mouse in each group after they are administered with Bacteroides fragilis. The numbers in the right quadrant represent the percentages of TCR γδ^{medium+} IFN-γ+ T cells and TCR γδ^{bright+} IFN-γ+ T cells. As can be seen from the flow cytometric analysis quadrant graph, the TCR γδ^{bright+} T cells show a significantly enhanced expression of IFN-γ, compared with TCR γδ^{medium+} T cells. FIG. 12 is a statistic analysis of expression of IFN-γ in TCR γδ^{medium+} T cells and TCR γδ^{bright+} T cells of melanoma-transplanted mice being administered with Bacteroides fragilis. As can be seen from the statistic graph, the TCR γδ^{bright+} T cell shows a significantly enhanced expression of IFN-γ compared to the TCR γδ^{medium+} T cells. In the statistical analysis graph, * represents student t-test p<0.05. p<0.05, the difference has statistical significance. There are twelve mice in each treatment group. It can be seen that the proportion of IFN-γ+ cells in TCR γδ^{bright+} T cells induced by Bacteroides fragilis increases significantly, compared to the TCR γδ^{medium+} T cells (FIGS. 11-12).

Similarly, for the melanoma-transplanted mice being administered with inactivated Bacteroides fragilis, the TCR γδ^{bright+} T cell shows a significantly enhanced expression of IFN-γ compared to the TCR γδ^{medium+} T cell (FIGS. 13-14).

Results of FIGS. 7-8 indicate that the Bacteroides fragilis and/or inactivated Bacteroides fragilis can induce a significant proliferation and accumulation of TCR γδ-strong positive (TCR γδ^{bright+}) T cells, and results of FIGS. 11-14 indicate that the TCR γδ-strong positive (TCR γδ^{bright+}) T cell shows a significantly stronger effector function of expressing IFN-γ compared to the TCR γδ-moderate positive (TCR γδ^{medium+}) T cells. Considering IFN-γ has an important anti-tumor and/or anti-infection function, these experiments manifest that administrating Bacteroides fragilis and/or inactivated Bacteroides fragilis can increase anti-tumor and/or anti-infection effect of γδ T cells.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A method of inducing at least one of a proliferation and an accumulation of γδ T cells, comprising: administering a drug comprising Bacteroides fragilis to a subject in need thereof.

2. A method of inducing at least one of a proliferation and accumulation of TCR γδ-strong positive T cells, comprising: administering a drug comprising Bacteroides fragilis to a subject in need thereof.

3. The method according to claim 1, wherein the Bacteroides fragilis is selected from any one of living Bacteroides fragilis; genetically recombined, altered or modified, attenuated, chemically treated, physically treated or inactivated Bacteroides fragilis; Bacteroides fragilis lysate; and/or Bacteroides fragilis culture supernatant.

4. A composition for inducing at least one of a proliferation and an accumulation of γδ T cells, comprising: Bacteroides fragilis as an active ingredient.

5. The composition according to claim 4, wherein the Bacteroides fragilis is selected from any one or more of living Bacteroides fragilis, genetically recombined, altered or modified, attenuated, chemically treated, physically treated or inactivated Bacteroides fragilis; Bacteroides fragilis lysate; and Bacteroides fragilis culture supernatant.

6. The composition according to claim 4, wherein the composition is any one of a pharmaceutical composition, a food, a health product, or a food additive.

7. A method of enhancing function of γδ T cells, comprising administering a drug comprising: Bacteroides fragilis to a subject in need thereof.

8. The method according to claim 7, wherein the Bacteroides fragilis is selected from any one or more of living Bacteroides fragilis; genetically recombined, altered or modified, attenuated, chemically treated, physically treated or inactivated Bacteroides fragilis; Bacteroides fragilis lysate; and Bacteroides fragilis culture supernatant.

9. A composition for enhancing function of γδ T cells, comprising Bacteroides fragilis as an active ingredient.

10. The composition according to claim 9, wherein the Bacteroides fragilis is selected from any one or more of living Bacteroides fragilis; genetically recombined, altered or modified, attenuated, chemically treated, physically treated or inactivated Bacteroides fragilis; Bacteroides fragilis lysate; and Bacteroides fragilis culture supernatant.

11. The method according to claim 2, wherein the Bacteroides fragilis is selected from any one or more of living Bacteroides fragilis; genetically recombined, altered or modified, attenuated, chemically treated, physically treated or inactivated Bacteroides fragilis; Bacteroides fragilis lysate; and Bacteroides fragilis culture supernatant.

12. The composition according to claim 5, wherein the composition is any one of a pharmaceutical composition, a food, a health product, or a food additive.
